(19) United States
(12) Patent Application Publication
(10) Pub. No.: US 2011/0212907 A1
(43) Pub. Date: Sep. 1, 2011

(54) HEXADECASACCHARIDES WITH ANTITHROMBOTIC ACTIVITY, INCLUDING A COVALENT BOND AND AN AMINO CHAIN

(75) Inventors: Marie-Claude Rouchon, Paris (FR); Joseph Schofield, Paris (FR); Dave Smith, Paris (FR); Patrick Soubayrol, Paris (FR)

(73) Assignee: SANOFI-AVENTIS, Paris (FR)

(21) Appl. No.: 13/060,745

(22) PCT Filed: Aug. 24, 2009
(86) PCT No.: PCT/FR2009/001024

§ 371 Date: May 20, 2011

(30) Foreign Application Priority Data
Aug. 26, 2008 (FR) 0804705

(51) Int. Cl. A61K 31/715; C08B 37/00; A61P 7/02; A61P 9/00; A61P 35/00; A61P 7/00

(52) U.S. Cl. 514/25; 536/53; 514/54; 536/17.5

(57) ABSTRACT

The present invention relates to novel synthetic hexadecasaccharides with antithrombotic activity, having at least one covalent bond with an amino chain, and to the preparation method thereof and to the therapeutic use thereof.

The present invention relates to novel synthetic hexadecasaccharides having at least one covalent bond with an amino chain and having the anticoagulant and antithrombotic pharmacological activities of heparin.

Patent application WO 2006/030104 describes synthetic hexadecasaccharides which have a covalent bond with biotin (hexahydro-2-oxo-1H-thieno[3,4-d]imidazole-4-pentanoic acid) or with a biotin derivative. Such hexadecasaccharides have an antithrombotic activity which makes them usable as anticoagulant agents and have, in addition, the advantage of being able to be rapidly neutralized with a specific antidote, in an emergency situation. This specific antidote is avidin (The Merck Index, Twelfth edition, 1996, M.N. 920, pages 151-152) or streptavidin, which are two tetrameric proteins of respective masses equal to approximately 66 000 and 60 000 Da, and which have a very strong affinity for biotin.

Novel hexadecasaccharide compounds which have structures analogous to those described in patent application WO 2006/030104, but which, in place of the covalent bond with biotin, have an amino chain, have now been identified. These novel saccharides advantageously have antithrombotic properties comparable to those described in the abovementioned patent application.

Generally, the invention therefore relates to synthetic hexadecasaccharides with antithrombotic activity, having at least one covalent bond with an amino chain of formula —NH—CO—(CH₂)₅—NH₂.

In particular, the subject of the present invention is the hexadecasaccharides of formula (I):

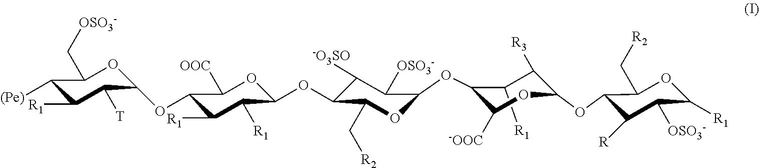

in which:
T represents an —NH—CO—(CH₂)₅—NH₂ group,
R represents a (C₁-C₆)alkoxy radical, in particular a methoxy radical or an —OSO₃⁻ radical,
R₁ represents a (C₁-C₆)alkoxy radical, in particular a methoxy radical or an —OSO₃⁻ radical,
R₂ represents a (C₁-C₆)alkoxy radical or an —OSO₃⁻ radical,
R₃ represents a (C₁-C₆)alkoxy radical, in particular a methoxy radical or an —OSO₃⁻ radical, or else R₃ constitutes an —O—CH₂— bridge, the —CH₂— group being bonded to the carbon atom bearing the carboxylic function on the same ring,
Pe represents a saccharide chain of following formula:

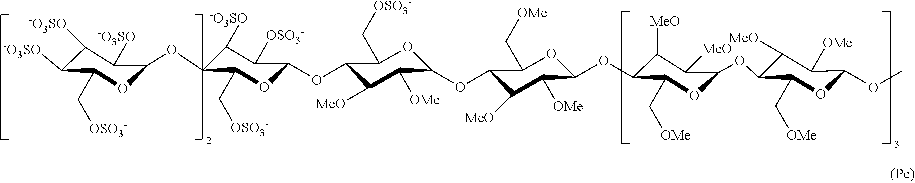

and also the pharmaceutically acceptable salts thereof.

The polysaccharide parts are constituted of uncharged and/or partially charged and/or totally charged alkylated monosaccharide units. The charged or uncharged units may be dispersed all along the chain or they may, on the contrary, be grouped into charged or uncharged saccharide domains.

In the present description, it has been chosen to represent the ¹C₄ conformation for L-iduronic acid and the ⁴C₁ conformation for D-glucuronic acid, but it is common knowledge that, in general, the conformation in solution of monosaccharide units fluctuates. Thus, L-iduronic acid may be of ⁴C₁²S₀ or ⁴C₁ conformation.

The invention encompasses the hexadecasaccharides in the acid form thereof or in the form of any one of the pharmaceutically acceptable salts thereof. In the acid form, the —COO⁻ and —SO₃⁻ functions are, respectively, in —COOH and —SO₃H form.

The expression “pharmaceutically acceptable salt of the polysaccharides of the invention” is intended to mean a polysaccharide in which one or more of the —COO⁻ and/or —SO₃⁻ functions is (are) ionically bonded to a pharmaceutically acceptable cation. The preferred salts according to the invention are those of which the cation is chosen from alkali metal cations, and even more preferably those of which the cation is Na⁺ or K⁺.

The compounds of formula (I) above also comprise those in which one or more hydrogen or carbon atoms have been replaced with the radioactive isotope thereof, for example tritium or carbon C¹⁴. Such labeled compounds are of use in research, metabolism or pharmacokinetic studies, or in biochemical tests as ligands.

In the context of the present invention, the term “(C₁-C₆)alkoxy radical” is intended to mean an —O-alkyl radical, the alkyl group being a linear or branched, saturated aliphatic radical having a chain of 1 to 6 carbon atoms. By way of example of alkyl radicals, mention may be made of methyl, ethyl, propyl, isopropylbutyl, isobutyl or tert-butyl radicals. By way of example of (C₁-C₆)alkoxy radicals, mention may be made of methoxy or ethoxy radicals.

According to another of its aspects, the present invention relates to the biotinylated hexadecasaccharides of general formula (I) in which:
R represents a methoxy radical or an —OSO₃⁻ radical,
R₁ represents a methoxy radical,
R₂ represents an —OSO₃⁻ radical, and
R₃ represents a methoxy radical.

According to another of its aspects, the invention relates to the following hexadecasaccharides:
methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-[N-(6-aminohexanoyl)]-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-3-O-methyl-2,6-di-O-sulfonato-α-D-glucopyranoside, sodium salt,
methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2, 3,6-tri-O-methyl-α-D-glucopyranosyl)-(1→4)-O-(2,3,6-tri-O-methyl-β-D-glucopyranosyl)-(1→4)]₃-(2-[N-(6-aminohexanoyl)]-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-2,3,6-tri-O-sulfonato-α-D-glucopyranoside, sodium salt.

In its principle, the method for preparing the compounds according to the invention uses di- or oligosaccharide base synthons prepared as previously reported in the literature. Reference will in particular be made to patents or patent applications EP 0 300 099, EP 0 529 715, EP 0 621 282 and EP 0 649 854 and also to the document C. van Boeckel, M. Petitou, Angew. Chem. Int. Ed. Engl., (1993), 32, pp. 1671-1690. These synthons are then coupled to one another so as to provide an entirely protected equivalent of a hexadecasaccharide according to the invention. This protected equivalent is then converted into a compound according to the invention.

One of the base synthons mentioned above contains a particular protected function allowing the subsequent introduction of the —CO—(CH₂)₅—NH₂ group (so as to form the —NH—CO—(CH₂)₅—NH₂ chain on the hexadecasaccharide), for example a latent amine function in azido group form or protected in N-phthalimido form.

In the coupling reactions mentioned above, a “donor” di- or oligosaccharide, activated on its anomeric carbon, reacts with an “acceptor” di- or oligosaccharide having a free hydroxyl.

The present invention relates to a method for preparing the compounds of formula (I), characterized in that:
in a first step, a completely protected equivalent of the desired hexadecasaccharide of formula (I) is obtained, containing a protected pentasaccharide precursor, bearing in particular an amine function which is suitably protected for the subsequent introduction of the —CO—(CH₂)₅—NH₂ group. This protected pentasaccharide precursor is itself extended by a protected precursor of the polysaccharide domain Pe;
in a second step, the negatively charged groups are introduced and/or unmasked;
in a third step, the amine function on the hexadecasaccharide is deprotected and then the protected —CO—(CH₂)₅—NH₂ group is introduced;
in a fourth step, the terminal NH₂ group is unmasked.

The synthesis of the pentasaccharide onto which the —CO—(CH₂)₅—NH₂ group will be grafted can be carried out according to the methods described in particular in the patent applications published under numbers WO 98/03554 and WO 99/36443, and also in the literature on polysaccharides.

The polysaccharide part which is the precursor of Pe is synthesized according to reactions well known to those skilled in the art, using oligosaccharide synthesis methods (G. J. Boons, Tetrahedron, (1996), 52, pp. 1095-1121, WO 98/03554 and WO 99/36443). Typically, a glycosidic-linkage donor oligosaccharide is coupled with a glycosidic-linkage acceptor oligosaccharide so as to produce another oligosaccharide, the size of which is equal to the sum of the sizes of the two reactive species. This sequence is repeated until the desired compound of formula (I) is obtained. The nature and the profile of the charge of the desired final compound determine the nature of the chemical entities used in the various steps of the synthesis, according to the rules well known to those skilled in the art. Reference may, for example, be made to C. van Boeckel, M. Petitou, Angew. Chem. Int. Ed. Engl., (1993), 32, pp. 1671-1690 or alternatively to H. Paulsen, “Advances in selective chemical syntheses of complex oligosaccharides” Angew. Chem. Int. Ed. Engl., (1982), 21, pp. 155-173.

The compounds of the invention are obtained from the completely protected polysaccharide precursors thereof using the following series of reactions:
the alcohol functions that must be converted into an O-sulfo group and the carboxylic acids are deprotected by removing the protective groups used during the production of the backbone,
the sulfo groups are then introduced,
the amine function of the hexadecasaccharide making it possible to introduce the —CO—(CH₂)₅—NH₂ group is deprotected,
the suitably protected —CO—(CH₂)₅—NH₂ group is introduced by means of a conventional amino/acid coupling reaction, then
the terminal —NH₂ group is unmasked.

The compounds of the invention can naturally be prepared using various strategies known to those skilled in the art of oligosaccharide synthesis.

The method described above is the preferred method of the invention. However, the compounds of formula (I) can be prepared by means of other methods that are well known in sugar chemistry, described, for example, in “Monosaccharides, Their Chemistry and their roles in natural products”, P. M. Collins and R. J. Ferrier, J. Wiley & Sons, (1995) and G. J. Boons, Tetrahedron, (1996), 52, p. 1095-1121.

The Pe pentasaccharides can therefore be obtained from disaccharide synthons in the manner described in the publication by C. van Boeckel, M. Petitou, Angew. Chem. Int. Ed. Engl., (1993), 32, 1671-1690.

Generally, the protective groups used in the method for preparing the compounds of formula I are those commonly used in sugar chemistry, as described, for example, in “Protective Groups in Organic Synthesis”, (1981), T W Greene, John Wiley & Sons, New York. The protective groups can, for example, be chosen from acetyl, halomethyl, benzoyl, levulinyl benzyl, substituted benzyl, optionally substituted trityl, tetrahydropyranyl, allyl, pentanyl, tert-butyldimethylsilyl (tBDMS) or trimethylsilylethyl groups.

The activating groups are those conventionally used in sugar chemistry according to, for example, G. J. Boons, Tetrahedron, (1996), 52, pp. 1095-1121. These activating groups are chosen, for example, from imidates, thioglycosides, pentenylglycosides, xanthates, phosphites or halides.

In particular, the introduction of the —CO—(CH₂)₅—NH₂ group onto the free amine function of the hexadecasaccharide, according to the method described above, can be carried out using a reactant of Act-CO—(CH₂)₅—NH-Pg type, in which “Act” represents an acid-function-activating group (such as an imide, for example a succinimide derivative, or else a mixed anhydride, or alternatively any other activating agent known in peptide chemistry for amino/acid coupling reactions) and “Pg” represents an amine-function-protecting group (such as a benzyloxycarbonyl group).

The method described above makes it possible to obtain the compounds of the invention in salt form. In order to obtain the corresponding acids, the compounds of the invention in salt form are brought into contact with an acid-form cation exchange resin. The compounds of the invention in acid form can then be neutralized with a base so as to obtain the desired salt. To prepare the salts of the compounds of formula (I), any inorganic or organic base which gives, with the compounds of formula (I), pharmaceutically acceptable salts can be used. Sodium hydroxide, potassium hydroxide, calcium hydroxide or magnesium hydroxide is preferably used as base. The sodium and calcium salts of the compounds of formula (I) are the preferred salts.

The invention will be understood more clearly by means of the detailed examples which follow, relating to the preparation of compounds according to the invention. These examples are not limiting and merely illustrate the present invention.

### EXAMPLE 1

### Preparation of Compound 1

Methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-[N-(6-aminohexanoyl]-2-deoxy-3-O-methyl-6-O-sulfonato-α-D-glucopyranosyl)-(1→4)-(2,3-di-O-methyl-β-O-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-3-O-methyl-2,6-di-O-sulfonato-α-D-glucopyranoside, sodium salt

### 1.1: Preparation of succinimidyl 6-(benzyloxycarbonyl amino)hexanoate

Triethylamine (0.63 ml, 4.52 mmol) is added to a solution of 6-(benzyloxycarbonylamino)hexanoic acid (1.00 g, 3.77 mmol) in dimethylformamide (20 ml) and the mixture is left to stir at ambient temperature and under argon for 30 minutes. The solution is cooled to 0° C. and ethyl chloroformate (0.43 ml, 4.52 mmol) is added dropwise. After two hours at ambient temperature, N-hydroxysuccinimide (0.52 g, 4.52 mmol) is added and the mixture is left to stir overnight at ambient temperature. The mixture is evaporated to dryness before taking up the residue in water to which ethyl acetate is added. The phases are separated and the aqueous phase is extracted with ethyl acetate. The organic phases are combined, dried over sodium sulfate, filtered and evaporated to dryness before purification on a silica gel column with an ethyl acetate/pentane mixture (75/25 v/v) as eluent. The fractions, once evaporated, give 1.13 g of succinimidyl 6(benzyloxycarbonylamino)hexanoate in the form of an oil. TLC: R_{f}=0.22 on silica gel plate with an n-heptane/ethyl acetate mixture (30/70 v/v) as eluent.

### 1.2: Preparation of Compound 1′

The amine chain is grafted onto the hexadecasaccharide 43, or methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-amino-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-2,3,6-tri-O-sulfonato-α-D-glucopyranoside, sodium salt, the preparation of which is described in patent application WO 2006/030104.

The compound 43 (200 mg, 40.6 μmol) is dissolved in a mixture of N,N-dimethylformamide (3 ml) and water (0.5 ml). A solution of succinimidyl 6-(benzyloxycarbonylamino)hexanoate (66 mg, 0.18 mmol) in N,N-dimethylformamide (0.7 ml) is added thereto dropwise. After stirring for 48 h at ambient temperature, the reaction volume is reduced to 1 ml by evaporation under vacuum and added dropwise to tert-butyl methyl ether (30 ml). The suspension is stirred for 1 h and filtered to give a solid product (190 mg), corresponding to compound 1′. It is compound 1 in which the amine function of the —NH—CO—(CH₂)₅—NH₂ group is protected with a benzyloxycarbonyl group.

Proton NMR at 200 MHz in deuterated water: the structure of the expected product is confirmed, owing to the fact that the spectrum obtained is identical to that produced on a product synthesized in accordance with example 2 of patent application WO 2006/030104, without the signals due to the atoms of the biotin part, but with signals of 5.2 to 5.3 and 7.4 to 7.5 ppm due to the benzyloxy group.

### 1.3: Preparation of Compound 1

The product 1′ obtained at the end of the preceding step (190 mg) is dissolved in deuterated water (6 ml). Palladium-on-carbon at 30% (19 mg) is added and the solution is left to stir at ambient temperature for 36 h under a hydrogen atmosphere. After filtration, the solution is added dropwise to 30 ml of tert-butyl methyl ether and the suspension is stirred for 1 h and filtered, to give 140 mg of compound 1.

TLC: R_{f}=0.8 on silica gel, using HPTLC plates from Merck grafted in the nitrile phase, with an acetonitrile/water/ethanol eluent [5/4/1 (v/v/v)].

Infrared on solid by total reflection method: 998.28; 1026,30; 1227.37; 1630.25; 2942,25; 3472.20 cm⁻¹.

Proton NMR at 600 MHz in deuterated water: the structure of the expected product is confirmed, owing to the fact that the spectrum obtained is identical to that produced on a product synthesized in accordance with example 2 of patent application WO 2006/030104, without the signals due to the atoms of the biotin part.

### EXAMPLE 2

### Preparation of Compound 2

Methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-2,3,6-tri-O-methyl-β-D-glucopyranosyl)-(1→4)]₃-(2-[N-(6-aminohexanoyl)]-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-3-O-methyl-2,6-di-O-sulfonato-α-D-glucopyranoside, sodium salt

The process as described in example 1 is carried out, starting from the hexadecasaccharide in 42, or methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-60 -D-glucopyranosyl)-(1→4)-(2,3,6-tri-O-methyl-(β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-amino-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-3-O-methyl-2,6-di-O-sulfonato-α-D-glucopyranoside, sodium salt, the preparation of which is described in patent application WO 2006/030104. 109 mg of compound 2 are obtained.

TLC: R_{f}=0.8 on silica gel, using HPTLC plates from Merck grafted in the nitrile phase, with an acetonitrile/water/ethanol eluent [5/4/1 (v/v/v)].

Infrared on solid by total reflection method: 997.79; 1025.99; 1226.97; 1634.37; 2937.94; 3468.92 cm⁻¹.

Proton NMR at 600 MHz in deuterated water: the structure of the expected product is confirmed, owing to the fact that the spectrum obtained is identical to that produced on a product synthesized in accordance with example 1 of patent application WO 2006/030104, without the signals due to the atoms of the biotin part.

### Compounds 42 and 43

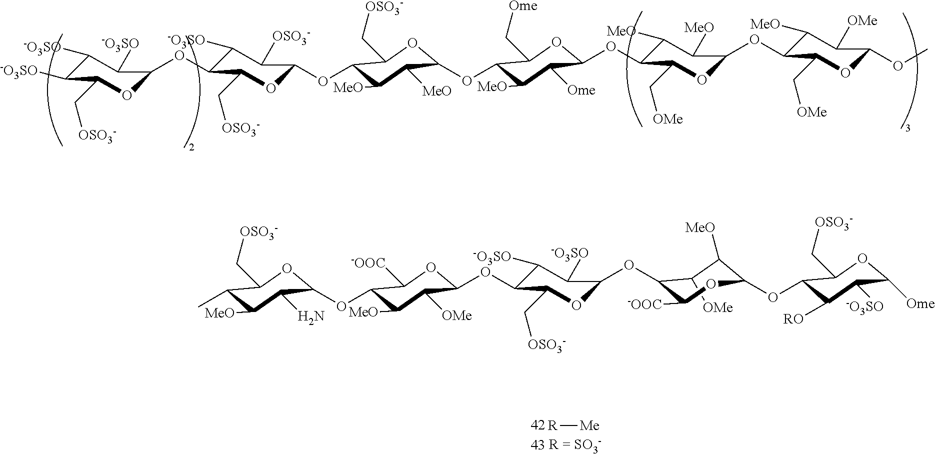

### Compound 1

### Example 1

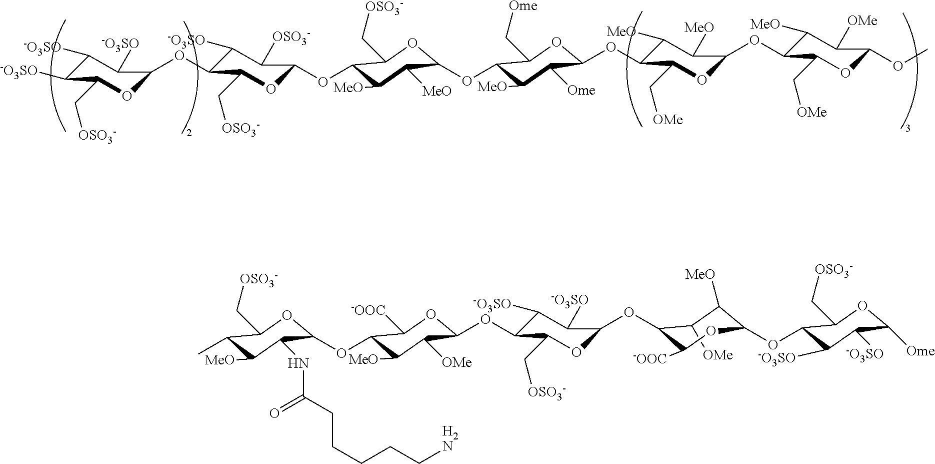

### Compound 2

### Example 2

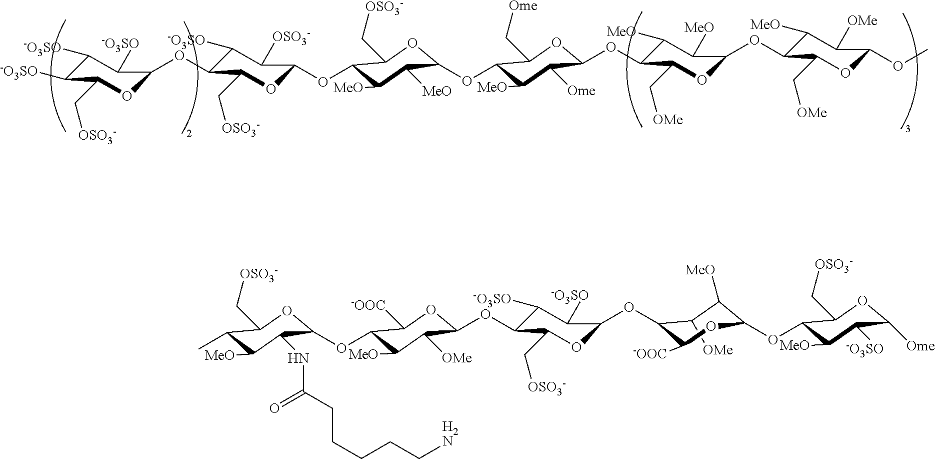

The compounds according to the invention were the subject of biochemical and pharmacological studies.

The pharmacological activity of these products was in particular studied in an in vitro model of inhibition of coagulation factor Xa, in the presence of antithrombin (anti-factor Xa activity dependent on the presence of antithrombin), as described by J.-M. Herbert et al., Blood, 1998, 91, 4197-4205. In this model, the antithrombotic properties of the compounds according to the invention are confirmed.

By virtue of their biochemical and pharmaceutical activity, the oligosaccharides of the present invention constitute very advantageous medicaments. Their toxicity is perfectly compatible with this use. They are also very stable and are therefore particularly suitable for constituting the active ingredient of patent medicines.

They can be used in various pathological conditions subsequent to a modification of the homeostasis of the coagulation system occurring in particular during cardiovascular and cerebrovascular system disorders, for instance thromboembolic disorders associated with atherosclerosis and with diabetes, such as unstable angina, stroke, post-angioplasty restenosis, endarterectomy, the insertion of endovascular prostheses; or thromboembolic disorders associated with post-thrombolysis rethrombosis, with infarction, with dementia of ischemic origin, with peripheral arterial disorders, with hemodialysis, with arterial fibrillation or else during the use of vascular prostheses for aortocoronary bypasses. These products can, moreover, be used for the treatment or prevention of thromboembolic pathological conditions of venous origin, such as pulmonary embolisms and deep vein thrombosis. They can be used either for preventing or for treating the thrombotic complications observed, for example, following surgical operations, the growth of tumors or coagulation disturbances induced by bacterial, viral or enzymatic activators. In the case of their use during the insertion of prostheses, the compounds of the present invention can cover prostheses and thus make them hemocompatible. In particular, they can be attached to intravascular prostheses (stents). In this case, they can optionally be chemically modified by introduction, at the nonreducing or reducing end, of an appropriate arm, as described according to EP 649 854. The compounds of the present invention can also be used as adjuvants during endarterectomy carried out with porous balloons.

The compounds according to the invention can be used for the preparation of medicaments intended for treating the above diseases.

According to another of the aspects of the present invention, a subject thereof is therefore a pharmaceutical composition containing, as active ingredient, a synthetic polysaccharide according to the invention or a pharmaceutically acceptable salt thereof, optionally in combination with one or more suitable inert excipients.

Said excipients are chosen according to the pharmaceutical form and the mode of administration desired: oral, sublingual, subcutaneous, intramuscular, intravenous, transdermal, transmucosal, local or rectal.

The active ingredient can also be provided in the form of a complex with a cyclodextrin, for example α-, β- or γ-cyclodextrin, 2-hydroxypropyl-β-cyclodextrin or methyl-β-cyclodextrin.

The active ingredient can also be released by a balloon containing it or by an endovascular expander inserted into the blood vessels. The pharmacological efficacy of the active ingredient is thus not affected.

In each dosage unit, the active ingredient is present in the amounts suitable for obtaining the desired prophylactic or therapeutic effect. Each dosage unit can contain from 0.1 to 100 mg of active ingredient, preferably 0.5 to 50 mg.

The compounds according to the invention can also be used in combination with one or more other active ingredients that are of use for the desired therapy, such as, for example, antithrombotics, anticoagulants, platelet aggregation inhibitors agents, for instance dipyridamole, aspirin, ticlopidine, clopidogrel, or glycoprotein IIb/IIIa complex antagonists.

1. A hexadecasaccharide of general formula (I): 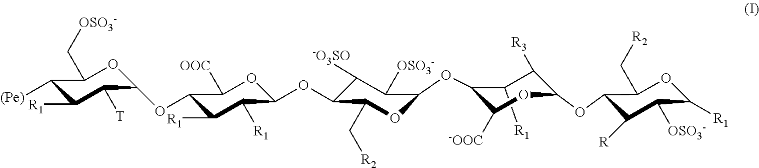
in which:
T represents an —NH—CO—(CH₂)₅—NH₂ group,
R represents a (C₁-C₆)alkoxy radical, in particular a methoxy radical or an —OSO₃⁻ radical,
R₁ represents a (C₁-C₆)alkoxy radical, in particular a methoxy radical or an —OSO₃— radical,
R₂ represents a (C ₁-C₆)alkoxy radical or an —OSO₃— radical,
R₃ represents a (C₁-C₆)alkoxy radical, in particular a methoxy radical or an —OSO₃⁻ radical, or else R₃ constitutes an —O—CH₂— bridge, the —CH₂— group being bonded to the carbon atom bearing the carboxylic function on the same ring,
Pe represents a saccharide chain of following formula: 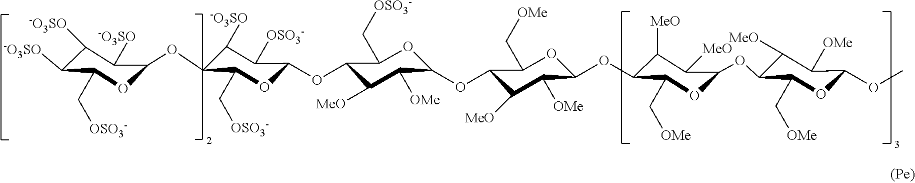
and also the pharmaceutically acceptable salts thereof.

2. The hexadecasaccharide as claimed in claim 1,
in which:
R represents a methoxy radical or an —OSO₃⁻ radical,
R₁ represents a methoxy radical,
R₂ represents an —OSO₃⁻ radical, and
R₃ represents a methoxy radical.

3. The hexadccasaccharides hexadecasaccharide as claimed in claim 1, comprising:
methyl (2,3,4,6-tetra-O-sulfonato-α-D-glucopyranosyl)-(1→4)-(2,3,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-[N-(6-aminohexanoyl)]-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-3-O-methyl-2,6-di-O-sulfonato-α-D-glucopyranoside, sodium salt,
methyl (2,3,4,6-tetra-O-sulfonato-α-D-glucopyranosyl)-(1→4)-(2,3 ,6-tri-O-sulfonato-α-D-glucopyranosyl)-(1→4)-(2,3,6-tri-O-sulfonato-β-D-glucopyranosyl)-(1→4)-(2,3-di-O-methyl-6-O-sulfonato-α-D-glucopyranosyl)-(1→4)-(2,3,6-tri-O-methyl-β-D-glucopyranosyl)-(1→4)-[(2,3,6-tri-O-methyl-α-D-glucopyranosyl)-(1→4)-O-(2,3,6-tri-O-methyl-β-D-glucopyranosyl)-(1→4)]₃-(2-[N-(6-aminohexanoyl)]-2-deoxy-3-O-methyl-6-O-sulfonato-α-D-glucopyranosyl)-(1→4)-(2,3-di-O-methyl-β-D-glucopyranosyluronic acid)-(1→4)-(2,3,6-tri-O-sulfonato-α-D-glucopyranosyl)-(1→4)-(2,3-di-O-methyl-α-L-idopyranosyluronic acid)-(1→4)-2,3,6-tri-O-sulfonato-α-D-glucopyranoside, sodium salt.

4. (canceled)

5. A pharmaceutical composition comprising, as an active ingredient, the hexadecasaccharide according to claim 1, and one or more suitable inert excipients.

6. A method of treating or preventing pathological conditions subsequent to a modification of the homeostasis of the coagulation system occurring during cardiovascular and cerebrovascular system disorders, for instance thromboembolic disorders associated with atherosclerosis and with diabetes, such as unstable angina, stroke, post-angioplasty restenosis, endarterectomy, the insertion of endovascular prostheses; or thromboembolic disorders associated with post-thrombolysis rethrombosis, with infarction, with dementia of ischemic origin, with peripheral arterial disorders, with hemodialysis, with arterial fibrillation, during the use of vascular prostheses for aortocoronary bypasses, in the treatment or prevention of thromboembolic pathological conditions of venous origin, such as pulmonary embolisms and deep vein thrombosis, for preventing or for treating thrombotic complications observed following surgical operations, the growth of tumors or coagulation disturbances induced by bacterial, viral or enzymatic activators in a patient in need thereof comprising administering to said patient a therapeutically effective amount of the pharmaceutical composition of claim 5.

7. (canceled)

8. (canceled)
